(19) United States
(12) Patent Application Publication
(10) Pub. No.: US 2021/0045443 A1
(43) Pub. Date: Feb. 18, 2021

(54) CONSUMABLE FOR SMOKING SUBSTITUTE DEVICE

(71) Applicant: Nerudia Limited, Liverpool (GB)

(72) Inventors: Chris Lord, Liverpool (GB); Pete Lomas, Liverpool (GB)

(21) Appl. No.: 17/087,130

(22) Filed: Nov. 2, 2020

Related U.S. Application Data
(63) Continuation of application No. PCT/EP2019/061141, filed on May 1, 2019.

(30) Foreign Application Priority Data
May 1, 2018 (GB) 1807153.0
Sep. 14, 2018 (GB) 1814990.6
Sep. 14, 2018 (GB) 1814991.4

(51) Int. Cl. A24F 40/42; A24F 40/10; A24F 40/44; A24F 40/48

(52) U.S. Cl. CPC A24F 40/42; A24F 40/48; A24F 40/44; A24F 40/10

(57) ABSTRACT

A consumable for a smoking substitute device comprises a liquid reservoir defining a volume which contains vapourisable liquid. Also disclosed are a smoking substitute device including the consumable, as well as methods of producing and using the consumable or smoking substitute device.

### CROSS-REFERENCE TO RELATED APPLICATIONS/INCORPORATION BY REFERENCE STATEMENT

The present patent application is a continuation of International Application No. PCT/EP2019/061141, filed May 1, 2019; which claims priority to the patent applications identified by GB Serial No. 1807153.0, filed May 1, 2018; GB Serial No. 1814990.6, filed Sep. 14, 2018; and GB Serial No. 1814991.4, filed Sep. 14, 2018. The entire contents of each of the above-referenced patent(s)/patent application(s) are hereby expressly incorporated by reference herein.

### FIELD OF THE INVENTION

The present invention relates to a method of preparing a consumable for a smoking substitute device, a consumable having a priming device, a method of filling a consumable, and a consumable having a liquid reservoir having less than 20% air by volume.

### BACKGROUND

The smoking of tobacco is generally considered to expose a smoker to potentially harmful substances. It is generally thought that a significant amount of the potentially harmful substances are generated through the heat caused by the burning and/or combustion of the tobacco and the constituents of the burnt tobacco in the tobacco smoke itself.

Combustion of organic material such as tobacco is known to produce tar and other potentially harmful by-products. There have been proposed various smoking substitute devices in order to avoid the smoking of tobacco.

Such smoking substitute devices can form part of nicotine replacement therapies aimed at people who wish to stop smoking and overcome a dependence on nicotine.

Smoking substitute devices, which may also be known as electronic nicotine delivery systems, may comprise electronic systems that permit a user to simulate the act of smoking by producing an aerosol, also referred to as a “vapour”, which is drawn into the lungs through the mouth (inhaled) and then exhaled. The inhaled aerosol typically bears nicotine and/or flavourings without, or with fewer of, the odour and health risks associated with traditional smoking.

In general, smoking substitute devices are intended to provide a substitute for the rituals of smoking, whilst providing the user with a similar experience and satisfaction to those experienced with traditional smoking and tobacco products.

The popularity and use of smoking substitute devices has grown rapidly in the past few years. Although originally marketed as an aid to assist habitual smokers wishing to quit tobacco smoking, consumers are increasingly viewing smoking substitute devices as desirable lifestyle accessories. Some smoking substitute devices are designed to resemble a traditional cigarette and are cylindrical in form with a mouthpiece at one end. Other smoking substitute devices do not generally resemble a cigarette (for example, the smoking substitute device may have a generally box-like form).

There are a number of different categories of smoking substitute devices, each utilising a different smoking substitute approach. A smoking substitute approach corresponds to the manner in which the substitute system operates for a user.

One approach for a smoking substitute device is the so-called “vaping” approach, in which a vapourisable liquid, typically referred to (and referred to herein) as “e-liquid”, is heated by a heating device to produce an aerosol vapour which is inhaled by a user. An e-liquid typically includes a base liquid as well as nicotine and/or flavourings. The resulting vapour therefore typically contains nicotine and/or flavourings. The base liquid may include propylene glycol and/or vegetable glycerin.

A typical vaping smoking substitute device includes a mouthpiece, a power source (typically a battery), a tank or liquid reservoir for containing e-liquid, as well as a heating device. In use, electrical energy is supplied from the power source to the heating device, which heats the e-liquid to produce an aerosol (or “vapour”) which is inhaled by a user through the mouthpiece.

Vaping smoking substitute devices can be configured in a variety of ways. For example, there are “closed system” vaping smoking substitute devices which typically have a sealed tank and heating element which is pre-filled with e liquid and is not intended to be refilled by an end user. One subset of closed system vaping smoking substitute devices include a main body which includes the power source, wherein the main body is configured to be physically and electrically coupled to a consumable including the tank and the heating element. In this way, when the tank of a consumable has been emptied, the main body can be reused by connecting it to a new consumable. Another subset of closed system vaping smoking substitute devices are completely disposable, and intended for one-use only.

There are also “open system” vaping smoking substitute devices which typically have a tank that is configured to be refilled by a user, so the device can be used multiple times.

An example vaping smoking substitute device is the myblu™ e-cigarette. The myblu™ e cigarette is a closed system device which includes a main body and a consumable. The main body and consumable are physically and electrically coupled together by pushing the consumable into the main body. The main body includes a rechargeable battery. The consumable includes a mouthpiece, a sealed tank which contains e-liquid, as well as a heating device, which for this device is a heating filament coiled around a portion of a wick which is partially immersed in the e-liquid. The device is activated when a microprocessor on board the main body detects a user inhaling through the mouthpiece. When the device is activated, electrical energy is supplied from the power source to the heating device, which heats e-liquid from the tank to produce a vapour which is inhaled by a user through the mouthpiece.

Another example vaping smoking substitute device is the blu PRO™ e-cigarette. The blu PRO™ e cigarette is an open system device which includes a main body, a (refillable) tank, and a mouthpiece. The main body and tank are physically and electrically coupled together by screwing one to the other. The mouthpiece and refillable tank are physically coupled together by screwing one into the other, and detaching the mouthpiece from the refillable tank allows the tank to be refilled with e-liquid. The device is activated by a button on the main body. When the device is activated, electrical energy is supplied from the power source to a heating device, which heats e-liquid from the tank to produce a vapour which is inhaled by a user through the mouthpiece.

The present inventor(s) have observed that the wick of the heating device may initially be dry, which can result in burning of the wick when the consumable is first used. Further the inventors have noted that, particularly during transportation, air present in the reservoir of the consumable may cause the consumable to leak. This is possibly due to the variations in temperature or atmospheric pressure experienced during transportation of the consumable.

The present invention has been devised in light of the above considerations.

### SUMMARY OF THE INVENTION

At its most general, embodiments of the present invention relate to: a method of preparing a consumable by priming the consumable to force vapourisable liquid from a liquid reservoir into a wick, a consumable including a priming device, a consumable having a liquid reservoir containing less than 20% air by volume, and a method of filling a liquid reservoir of a consumable such that it has less than 20% air by volume.

According to a first aspect of the present invention, there is provided a consumable for a smoking substitute device, said consumable comprising: a liquid reservoir, for storing vaporisable liquid; an outlet, at least partially located within the liquid reservoir; and a solid air-displacing element, located within the liquid reservoir, and which is configured to fill at least 8% of a volume of the liquid reservoir.

By displacing the air which would otherwise be in the liquid reservoir the risk of leakage due to environmental changes is diminished. By a volume, it may be meant the free volume (fillable with liquid) plus the volume of the solid air-displacing element and that of this total volume the solid air-displacing element is configured to fill at least 8%. By configured to fill, it may be meant that the solid air-displacing element has such external dimensions so as to fill at least 8% of the volume.

Optionally, consumable may comprise a heater assembly, at least partially located within the liquid reservoir and sealed to the outlet via a seal.

Advantageously, an interior of the heater assembly may be fluidly connected to the liquid reservoir.

Conveniently, the outlet may be fluidly connected to the interior of the heater assembly.

According to a second aspect of the present invention, there is provided a smoking substitute device including the consumable according to the first aspect.

Optionally, the liquid reservoir may be referred to as a tank.

Advantageously, the heater assembly may be a coil and wick assembly.

Conveniently, the solid air-displacing element may be the seal, sealing the outlet to the heater assembly.

Optionally, the air-displacing element may fill no more than 20% of the volume of the liquid reservoir.

Advantageously, solid air-displacing element may be formed of silicone.

Conveniently, the liquid reservoir may have a free volume of 1.7 ml, and the seal has have a volume of greater than 0.13 ml and no more than 0.33 ml. By free volume, it is meant the volume of the liquid reservoir which is fillable with liquid e.g. not including any solid parts contained therein.

Optionally, the liquid reservoir may contain vaporisable liquid

Advantageously, the liquid reservoir may contain substantially no air. Any air that may be present within the liquid reservoir, when the liquid reservoir contains vaporisable liquid, may be of a negligible amount and, for example, does not form a visible air bubble.

Conveniently, the liquid reservoir may contain 1.5 ml of vaporisable liquid.

Optionally, the solid air-displacing element may be located proximal to the heater assembly.

Advantageously, the solid air-displacing element may extend from the position proximal to the heater assembly along an axis of the outlet.

Conveniently, the axis of the outlet may lie along a line joining the heater assembly and a mouthpiece of the consumable.

Optionally, the outlet may fluidly connect the heater assembly to a mouthpiece located at an opposite end of the consumable to the heater assembly.

Advantageously, the solid air-displacing element may extend along at least 20% of the length of the outlet. By doing so, it can be ensured that the flow of vaporisable liquid within the liquid reservoir to the heater assembly is not impeded

Conveniently, the solid air-displacing element may be located on an opposite side of the heater assembly to an air inlet of the consumable.

Optionally, the solid air-displacing element may be toroidal in shape.

Advantageously, the outlet may be present within the hole of the toroidal shape solid air-displacing element.

According to a third aspect of the present invention, there is provided a consumable for a smoking substitute device, comprising: a liquid reservoir, defining a volume which contains vapourisable liquid; wherein the liquid reservoir contains less than 20% air by volume.

Conveniently, the liquid reservoir may contain less than 10% air by volume. Optionally, the liquid reservoir may contain less than 5% air by volume. Advantageously, the liquid reservoir may contain less than 3% air by volume. Conveniently, the liquid reservoir may contain less than 2% air by volume. Optionally, the liquid reservoir may contain substantially no air.

Advantageously, the liquid reservoir may contain 1.5 ml of vaporisable liquid.

Conveniently, the consumable may comprise a solid air-displacing element, located within the liquid reservoir, and which is configured to fill at least 8% of a volume of the liquid reservoir.

Optionally, the solid air-displacing element may be configured to fill no more than 20% of the volume of the liquid reservoir.

Advantageously, the solid air-displacing element may be formed of silicone.

Conveniently, the solid air-displacing element may be toroidal in shape.

Optionally, the solid air-displacing element may have a volume of greater than 0.13 ml and no more than 0.33 ml.

Advantageously, the consumable may further comprise: an outlet (306), at least partially located within the liquid reservoir; and a heater assembly, at least partially located within the liquid reservoir and sealed to the outlet via a seal.

Conveniently, the outlet may fluidly connect the heater assembly to a mouthpiece located at an opposite end of the consumable to the heater assembly.

Optionally, the outlet may be present within the hole of the toroidal shape solid air-displacing element.

Advantageously, the solid air-displacing element may be located proximal to the heater assembly.

Conveniently, the solid air-displacing element may be the seal, sealing the outlet to the heater assembly.

Optionally, the solid air-displacing element may be located on an opposite side of the heater assembly to an air inlet of the consumable.

Advantageously, the solid air-displacing element may extend from the position proximal to the heater assembly along an axis of the outlet.

Conveniently, the solid air-displacing element may extend along at least 20% of the length of the outlet.

According to a fourth aspect of the present invention, there is provided a smoking substitute device including the consumable according to the third aspect.

According to a fifth aspect of the present invention, there is provided a method of filling a consumable for a smoking substitute device, the method comprising adding vapourisable liquid to a liquid reservoir of the smoking substitute device, wherein after filing the consumable, the liquid reservoir contains less than 20% air by volume.

Optionally, the method of filling may further comprise adding a solid air-displacing element to the liquid reservoir.

Advantageously, the method may comprise adding the vapourisable liquid before adding the solid air-displacing element.

Conveniently, after filing the consumable, the liquid reservoir may contain less than 10% air by volume. Optionally, after filing the consumable, the liquid reservoir may contain less than 5% air by volume. Advantageously, after filing the consumable, the liquid reservoir may contain less than 3% air by volume. Conveniently, after filing the consumable, the liquid reservoir may contain less than 2% air by volume. Optionally, after filing the consumable, the liquid reservoir may contain less than 1% air by volume.

According to a sixth aspect of the invention, there is provided a method of preparing a consumable for a smoking substitute device, the consumable comprising: a liquid reservoir defining a volume which contains vapourisable liquid; and a heater assembly comprising a wick, wherein the method comprises priming the consumable to force vapourisable liquid from the liquid reservoir into the wick.

Burning of the wick can occur when a dry wick is heated, and results in the user receiving an unpleasant taste. Priming the pod to force liquid into the wick in this way reduces the chances of burning the wick. This is particularly applicable to situations in which the liquid reservoir is substantially full of liquid and contains little air, which may result in the formation of a vacuum within the liquid reservoir, preventing flow of the vapourisable liquid into the wick.

Optionally, the consumable may comprise deformable walls, the priming the consumable comprising squeezing the deformable walls to force vapourisable liquid into the wick. Squeezing the deformable walls causes a pressure increase in the liquid reservoir, forcing the vapourisable liquid into the wick. Upon release, a backflow of air into the liquid reservoir occurs. The air bubbles to the top of the e-liquid tank, removing the vacuum created without the priming.

Advantageously, the liquid reservoir may be a tank, the deformable walls forming walls of the tank.

Conveniently, the consumable may comprise a button, wherein the priming comprises pushing the button to squeeze the deformable walls.

Optionally, the button may comprise a window permitting a level of vapourisable liquid in the liquid reservoir to be visually assessed.

Advantageously, the priming may comprise squeezing the deformable walls at least three times.

Conveniently, the priming may comprise squeezing the deformable walls when the consumable is connected to a main body of the smoking substitute device, the consumable and the main body configured to prevent oversqueezing of the deformable walls. This may be achieved by the button extending through a slot of the main body when the consumable is connected to the main body, the main body preventing the user being able to push the button to the extent that the deformable walls are oversqueezed. Oversqueezing of the deformable walls may result in leakage of vapourisable liquid out of the bottom of the consumable.

Optionally, the liquid reservoir may contain less than 5% air by volume. The liquid reservoir containing a smaller volume of air assists in forcing vapourisable liquid into the wick. This is because squeezing a consumable (or reducing a volume of the liquid reservoir of the consumable in some other way) with a large volume of air primarily causes compression of the air, with only a small amount of vapourisable liquid flowing into the wick. When the volume of air in the liquid reservoir is low, the priming causes a smaller change in volume of air, resulting in more vapourisable liquid being forced into the wick.

Advantageously, the liquid reservoir may contain less than 3% air by volume.

Conveniently, the liquid reservoir may contain less than 1% air by volume.

Optionally, the liquid reservoir may contain substantially no air.

Advantageously, the liquid reservoir may contain 1.5 ml of vaporisable liquid.

Conveniently, the consumable may further comprise a solid air-displacing element, located within the liquid reservoir, and wherein the solid air-displacing element fills at least 8% of a volume of the liquid reservoir.

According to a seventh aspect of the invention, there is provided a consumable for a smoking substitute device, the consumable comprising: a liquid reservoir defining a volume for storing vapourisable liquid; a heater assembly comprising a wick; and a priming device actuatable to force vapourisable liquid from the liquid reservoir into the wick. The consumable may comprise any of the optional, advantageous and convenient features described above.

According to an eighth aspect of the invention, there is provided a smoking substitute device comprising the consumable of the sixth aspect.

The invention includes the combination of the aspects and preferred features described except where such a combination is clearly impermissible or expressly avoided.

### SUMMARY OF THE FIGURES

So that the invention may be understood, and so that further aspects and features thereof may be appreciated, embodiments illustrating the principles of the invention will now be discussed in further detail with reference to the accompanying figures, in which:

FIG. 1(a) shows an example smoking substitute device;

FIG. 1(b) shows the main body of the smoking substitute device of FIG. 1(a) without the consumable;

FIG. 1(c) shows the consumable of the smoking substitute device of FIG. 1(a) without the main body;

FIG. 2(a) is a schematic view of the main body of the smoking substitute device of FIG. 1(a);

FIG. 2(b) is a schematic view of the consumable of the smoking substitute device of FIG. 1(b);

FIG. 3 is a cross-sectional view of a consumable of the smoking substitute device.

FIG. 4 is a cross-sectional view of the consumable of the smoking substitute device containing liquid.

FIG. 5 is a perspective view of the consumable.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

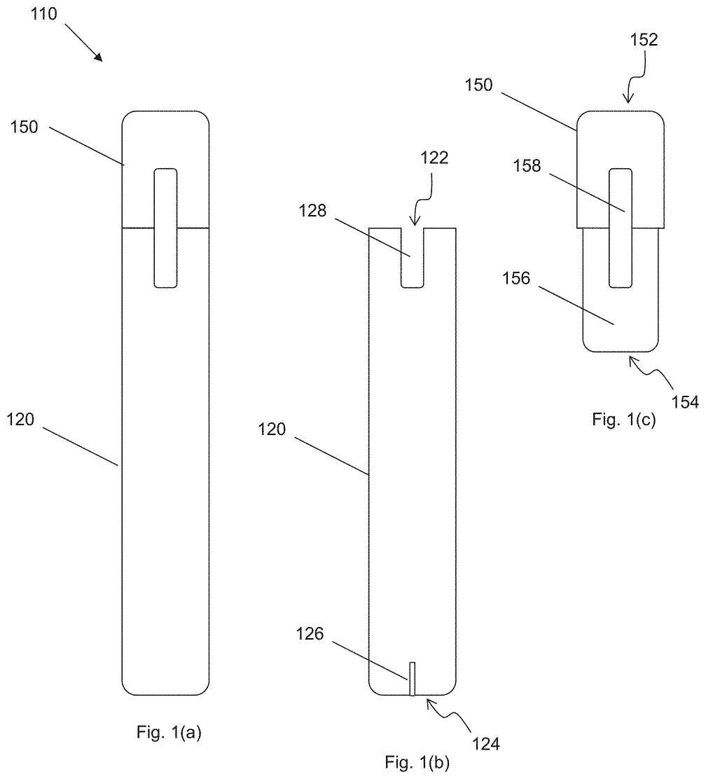

FIG. 1(a) shows an example smoking substitute device 110. In this example, the smoking substitute device 110 includes a main body 120 and a consumable 150. The consumable 150 may alternatively be referred to as a “pod”. The consumable may also be referred to as a cartridge or cartomizer.

In this example, the smoking substitute device 110 is a closed system vaping device, wherein the consumable 150 includes a sealed tank 156, which defines a liquid reservoir and is intended for one-use only.

FIG. 1(a) shows the smoking substitute device 110 with the main body 120 physically coupled to the consumable 150.

FIG. 1(b) shows the main body 120 of the smoking substitute device 110 without the consumable 150.

FIG. 1(c) shows the consumable 150 of the smoking substitute device 110 without the main body 120.

The main body 120 and the consumable 150 are configured to be physically coupled together, in this example by pushing the consumable 150 into an aperture in a top end 122 of the main body 120. In other examples, the main body 120 and the consumable could be physically coupled together by screwing one onto the other, or through a bayonet fitting, for example. An optional light 126, e.g. an LED located behind a small translucent cover, is located a bottom end 124 of the main body 120. The light 126 may be configured to illuminate when the smoking substitute device 110 is activated.

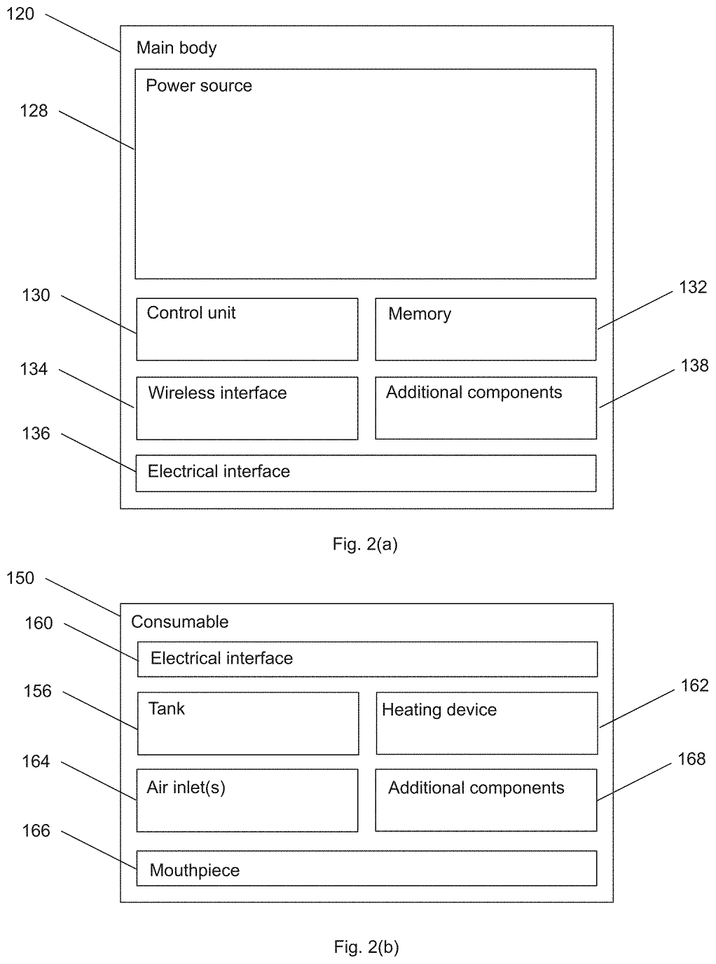

The consumable 150 includes a mouthpiece (not shown) at a top end 152 of the consumable 150, as well as one or more air inlets (not shown in FIG. 2) so that air can be drawn into the smoking substitute device 110 when a user inhales through the mouthpiece. At a bottom end 154 of the consumable 150, there is located a tank 156 that contains e-liquid. The tank 156 may be a translucent body, for example.

The tank 156 preferably includes a window 158, so that the amount of e-liquid in the tank 156 can be visually assessed. The main body 120 includes a slot 128 so that the window 158 of the consumable 150 can be seen whilst the rest of the tank 156 is obscured from view when the consumable 150 is inserted into the aperture in the top end 122 of the main body 120.

The tank 156 may be referred to as a “clearomizer” if it includes a window 158, or a “cartomizer” if it does not.

The consumable 150 may identify itself to the main body 120, via an electrical interface, RFID chip, or barcode.

FIG. 2(a) is a schematic view of the main body 120 of the smoking substitute device 110.

FIG. 2(b) is a schematic view of the consumable 150 of the smoking substitute device 110.

As shown in FIG. 2(a), the main body 120 includes a power source 140, a control unit 130, a memory 132, a wireless interface 134, an electrical interface 136, and, optionally, one or more additional components 138.

The power source 140 is preferably a battery, more preferably a rechargeable battery.

The control unit 130 may include a microprocessor, for example.

The memory 132 is preferably includes non-volatile memory. The memory may include instructions which, when implemented, cause the control unit 130 to perform certain tasks or steps of a method.

The wireless interface 134 is preferably configured to communicate wirelessly with the mobile device 2, e.g. via Bluetooth®. To this end, the wireless interface 134 could include a Bluetooth® antenna. Other wireless communication interfaces, e.g. WiFi®, are also possible. As discussed above, the wireless interface 134 may be configured to communicate wirelessly with the remote server 2.

The electrical interface 136 of the main body 120 may include one or more electrical contacts. The electrical interface 136 may be located in, and preferably at the bottom of, the aperture in the top end 122 of the main body 120. When the main body 120 is physically coupled to the consumable 150, the electrical interface 136 may be configured to pass electrical power from the power source 140 to (e.g. a heating device of) the consumable 150 when the smoking substitute device 110 is activated, e.g. via the electrical interface 160 of the consumable 150 (discussed below). When the main body 120 is not physically coupled to the consumable 150, the electrical interface may be configured to receive power from the charging station 6. The electrical interface 136 may also be used to identify the consumable 150 from a list of known consumables. For example, the consumable may be a particular flavour and/or have a certain concentration of nicotine. This can be identified to the control unit 130 of the main body 120 when the consumable is connected to the main body. Additionally, or alternatively, there may be a separate communication interface provided in the main body 120 and a corresponding communication interface in the consumable 150 such that, when connected, the consumable can identify itself to the main body 120.

The additional components 138 of the main body 120 may include the optional light 126 discussed above.

The additional components 138 of the main body 120 may, if the power source 140 is a rechargeable battery, include a charging port configured to receive power from the charging station 6. This may be located at the bottom end 124 of the main body 120. Alternatively, the electrical interface 136 discussed above is configured to act as a charging port configured to receive power from the charging station 6 such that a separate charging port is not required.

The additional components 138 of the main body 120 may, if the power source 140 is a rechargeable battery, include a battery charging control circuit, for controlling the charging of the rechargeable battery. However, a battery charging control circuit could equally be located in the charging station 6 (if present).

The additional components 138 of the main body 120 may include an airflow sensor for detecting airflow in the smoking substitute device 110, e.g. caused by a user inhaling through a mouthpiece 166 (discussed below) of the smoking substitute device 110. The smoking substitute device 110 may be configured to be activated when airflow is detected by the airflow sensor. This optional sensor could alternatively be included in the consumable 150 (though this is less preferred where the consumable 150 is intended to be disposed of after use, as in this example). The airflow sensor can be used to determine, for example, how heavily a user draws on the mouthpiece or how many times a user draws on the mouthpiece in a particular time period.

The additional components 138 of the main body 120 may include an actuator, e.g. a button. The smoking substitute device 110 may be configured to be activated when the actuator is actuated. This provides an alternative to the airflow sensor noted, as a mechanism for activating the smoking substitute device 110.

As shown in FIG. 2(b), the consumable 150 includes the tank 156, an electrical interface 160, a heating device 162, one or more air inlets 164, a mouthpiece 166, and, optionally, one or more additional components 168.

The electrical interface 160 of the consumable 150 may include one or more electrical contacts. The electrical interface 136 of the main body 120 and an electrical interface 160 of the consumable 150 are preferably configured to contact each other and thereby electrically couple the main body 120 to the consumable 150 when the main body 120 is physically coupled to the consumable 150. In this way, electrical energy (e.g. in the form of an electrical current) is able to be supplied from the power source 140 in the main body 120 to the heating device 162 in the consumable 150.

The heating device 162 is preferably configured to heat e-liquid contained in the tank 156, e.g. using electrical energy supplied from the power source 140. In one example, the heating device 162 may include a heating filament and a wick, wherein a first portion of the wick extends into the tank 156 in order to draw e-liquid out from the tank 156, and wherein the heating filament coils around a second portion of the wick located outside the tank 156. In this example, the heating filament is configured to heat up e-liquid drawn out of the tank 156 by the wick to produce an aerosol vapour.

The one or more air inlets 164 are preferably configured to allow air to be drawn into the smoking substitute device 110, when a user inhales through the mouthpiece 166.

In use, a user activates the smoking substitute device 110, e.g. through actuating an actuator included in the main body 120 or by inhaling through the mouthpiece 166 as described above. Upon activation, the control unit 130 may supply electrical energy from the power source 140 to the heating device 162 (via electrical interfaces 136, 166), which may cause the heating device 162 to heat e-liquid drawn from the tank 156 to produce a vapour which is inhaled by a user through the mouthpiece 166.

As an example of one of the one or more additional components 168, an interface for obtaining an identifier of the consumable may be provided. As discussed above, this interface may be, for example, an RFID reader, a barcode or QR code reader, or an electronic interface which is able to identify the consumable to the main body. The consumable may, therefore include any one or more of an RFID chip, a barcode or QR code, or memory within which is an identifier and which can be interrogated via the electronic interface in the main body.

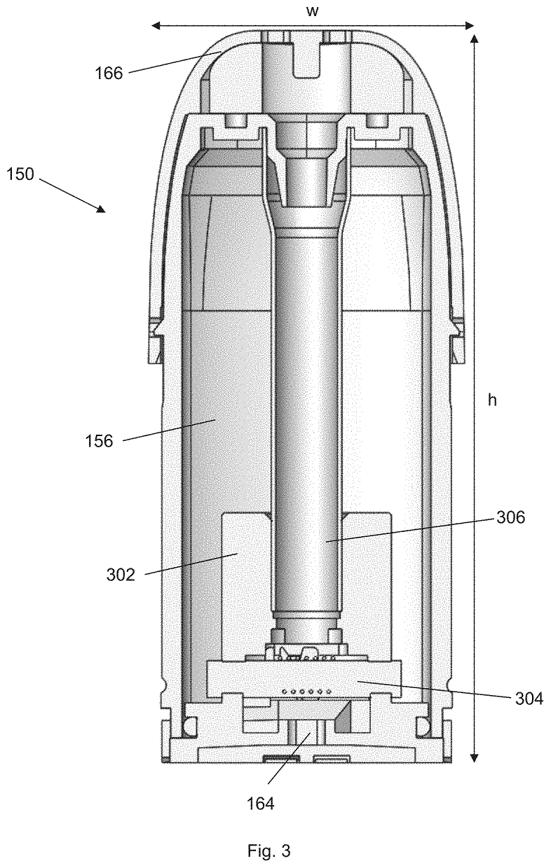

Of course, a skilled reader would readily appreciate that the smoking substitute device 110 shown in FIGS. 2 and 3 shows just one example implementation of a smoking substitute device, and that other forms of smoking substitute device could be used.

As another example, an entirely disposable (one use) smoking substitute device could be used as the smoking substitute device.

FIG. 3 shows a cross-sectional view of a consumable 150 according to the present invention. Broadly, the consumable comprises a tank 156, which defines a liquid reservoir and within which is provided: a solid air-displacing element 302; a heater assembly, in this example coil and wick assembly 304; and an outlet 306. The outlet fluidly connects the coil and wick assembly to the mouthpiece 166.

The tank is provided by an outer casing of the consumable. The coil and wick assembly 304 broadly comprises an outer shell with one or more apertures. These apertures are filled with a wick material, so that liquid may only ingress the coil and wick assembly from the tank via capillary action. The wick material passes through or proximal to a coil, which is connected to one or more electrical contacts. By providing an electrical current to the one or more contacts, the coil may be heated and so the liquid within the wick material is vaporised. The coil and wick assembly 304 is connected to the air inlet 164 of the consumable, so that when the user draws on the mouthpiece, a flow of air passes from the air inlet through the coil and wick assembly thereby drawing with it vaporised liquid. This mixed flow of air and vaporised liquid then passes up the outlet 306, in this example a chimney or tube, before exiting the consumable 150 via mouthpiece 166. It is important then that liquid may only enter the coil and wick assembly via the one or more apertures and then, only via the wick.

The solid air-displacing element 302 is, in this example, a silicone seal which seals the coil and wick assembly 304 to the outlet 306. This seal ensures that liquid within the tank 156 cannot pass directly into the outlet 306. Instead, in use, vaporisable liquid is wicked into the coil and wick assembly by the wick, e.g. via capillary action, and vaporised by the coil within. Therefore the outlet 306 should only receive vaporised liquid.

The solid air-displacing element 302 is provided with a volume such that when the tank is filled with a prescribed amount of liquid, there is no or substantially no air left in the tank. The presence of air in the tank is linked with an increased risk of leakage, either during normal use or in transit. For example, an increase in the temperature of the consumable as a whole may lead to expansion of any air within the tank. This can increase the pressure exerted by the liquid on the interior components of the tank, and some liquid may egress. As an alternative example, a decrease in atmospheric pressure (for example, as experienced in the hold of an aircraft) may similarly lead to an increase in the pressure exerted by the liquid on the interior components of the tank as any air contained therein may expand.

In one example, the tank may have an initial free volume (i.e. fillable with liquid, and not inclusive of the solid air-displacing element) of around 1.83 ml. As it is preferred that consumables contain around 1.5 ml of vaporisable liquid, this leaves around 0.33 ml of free space in which air may be present. By providing a solid air-displacing element 302 with a 0.33 ml volume, this 0.33 ml of free space is now displaced by the solid air-displacing element 302.

To fill the consumable, the vapourisable liquid is added to the liquid reservoir. In this example, 1.5 ml of vapourisable liquid is added. After adding the vapourisable liquid, the solid air-displacing element 302 is inserted into the liquid reservoir. The solid air-displacing element 302 displaces some of the vapourisable liquid.

The result is a tank which is substantially filled with liquid, and substantially no gas. In this example, as the solid air-displacing element 302 has a volume of around 0.33 ml, it occupies around 18% of the initial free volume (i.e. 1.83 ml). Said another way, by providing a 0.33 ml solid air-displacing element 302 in the 1.83 ml initially free volume, the final free volume (filled with liquid) is 1.5 ml. This means that the liquid reservoir contains substantially no air by volume.

In other examples, the liquid reservoir contains less than 20% air by volume. In other examples, the liquid reservoir contains less than 10% air by volume. In other examples, the liquid reservoir contains less than 5% air by volume. In other examples, the liquid reservoir contains less than 3% air by volume. In other examples, the liquid reservoir contains less than 2% air by volume. In other examples, the liquid reservoir contains less than 1% air by volume.

To put this another way, in other examples, at least 80% of the final free volume of the liquid reservoir is vapourisable liquid. In other examples, at least 90% of the final free volume of the liquid reservoir is vapourisable liquid. In other examples, at least 95% of the final free volume of the liquid reservoir is vapourisable liquid. In other examples, at least 97% of the final free volume of the liquid reservoir is vapourisable liquid. In other examples, at least 98% of the final free volume of the liquid reservoir is vapourisable liquid. In other examples, at least 99% of the final free volume of the liquid reservoir is vapourisable liquid. In other examples, substantially 100% of the final free volume of the liquid reservoir is vapourisable liquid.

By reducing the amount of air in the liquid reservoir, the risk of leakage due to environmental changes is reduced. This is particularly important during transportation when the external pressure falls (for example, on an aircraft), because, when air is present in the reservoir, such a fall in external pressure causes expansion of the air and ejection of vapourisable liquid from the liquid reservoir.

The tank in this example has a height, h, of around 32 mm and a width, w, of around 14 mm. The depth of the tank (as measured) in a direction perpendicular to the width and height, is around 7 mm. The tank in this example is elliptical and curved.

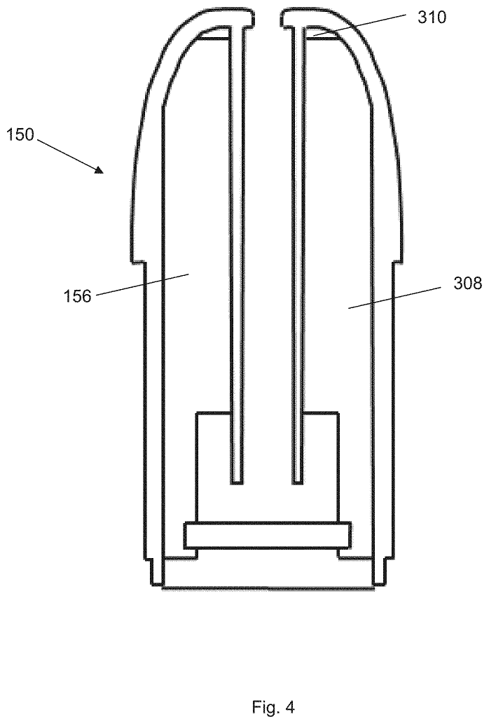

FIG. 4 shows the consumable 150 in which the tank 156 comprises liquid 308 and air 310. In this example, the tank 156 contains less than 1% air 310 by volume, and more than 99% liquid 308 by volume. As described above, in other examples the tank 156 contains substantially no air.

While the examples described here uses a solid air displacing element to reduce the amount of air in the liquid reservoir, there are other options for achieving this. For example, the liquid reservoir could be carefully filled with liquid.

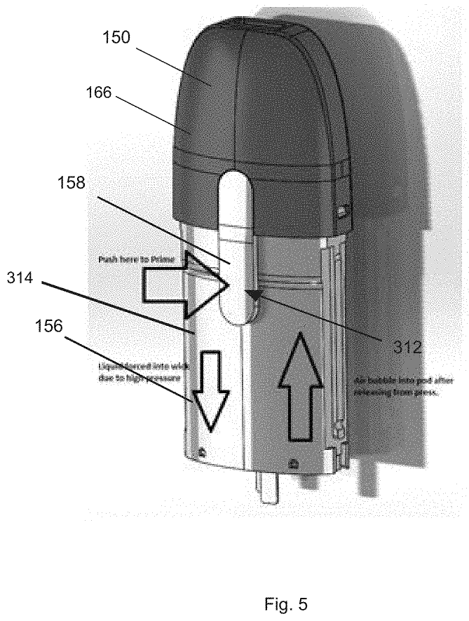

FIG. 5 is a perspective view of the consumable 150 demonstrating a method of preparing the consumable 150 ready for use with a smoking substitute device.

In addition to the features described above with respect to FIG. 1 to FIG. 4, the consumable 150 comprises a priming device 312. The priming device 312 comprises deformable walls 314 of the consumable 150. The deformable walls 314 define the liquid reservoir 156, which in this example is the tank 156. The deformable walls 314 are front and rear walls of the consumable 150, below the mouthpiece 166 in use.

The priming device 312 further comprises a button 158. The button 158 is a portion of the consumable 150 arranged such that pushing of the button causes squeezing of the deformable walls 314 inwardly towards one another so as to reduce the volume of space therebetween. The button 158 is integrally formed with the deformable walls 314. In this example, the button 158 forms the same component as the window 158 described above, such that the button is translucent, permitting a level of vapourisable liquid in the tank 156 to be visually assessed. The button 158 is located in the consumable such that the button 158 extends into the main body 120 (protruding through the slot 128 of the main body 120, as shown in FIG. 1). The main body 120 therefore limits the extent that the button 158 can be pressed by the user, due to limited accessibility to the button 158 through the slot 128, preventing oversqueezing of the deformable walls 314.

As described above, the tank 156 is filled so that it contains a small amount of air. In this example, the tank 156 contains substantially no air. This means that after filling of the tank 156, a vacuum forms in the tank 156, preventing flow of vapourisable liquid into the wick.

After transportation, (for example, after a user has received the consumable 150), the user primes the consumable to force vapourisable liquid from the tank 156 into the wick. In order to do this, the user squeezes the deformable walls 314, which increases the pressure in the liquid reservoir 156, forcing the vapourisable liquid into the wick. The user squeezes the walls by squeezing the button 158.

The user subsequently releases the deformable walls 314, causing the deformable walls 314 to resiliently return to their undeformed position. This results in a pressure drop within the liquid reservoir 156. The pressure drop effects backflow of air into the liquid reservoir, reducing the vacuum in the tank 156. Since the wick is in fluid communication with air in the outlet 306 air backflow occurs preferentially to back flow of liquid in the wick, since the pressure in the outlet 306 is relatively high. The vapourisable liquid is also held in the wick by capillary forces.

To ensure the wick is well saturated with vapourisable liquid, the user squeezes the deformable walls 314 at least three times. The user need not squeeze the deformable walls 314 more than five times. Once the consumable 150 has been primed, the user activates the smoking substitute device 110 as described above.

In other examples, the priming device does not comprise deformable walls, and is instead implemented by another device which enables the volume of the liquid reservoir to be decreased or the e-liquid pressure to be increased (for example, using a plunger arrangement to reduce a volume of the liquid reservoir).

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

### LIST OF FEATURES

110 Smoking substitute device
120 Main body
122 Top end of main body
124 Bottom end of main body
126 Light
128 Slot
130 Control unit
132 Memory
134 Wireless interface
136 Electrical interface
138 Additional component
140 Power source
150 Consumable
152 Top end of consumable
154 Bottom end of consumable
156 Tank
158 Window
160 Electrical interface
162 Heating device
164 Air inlets
166 Mouthpiece
168 Additional components
302 Solid air-displacing element
304 Coil and wick assembly
306 Outlet
308 Liquid
310 Air
312 Priming device
314 Deformable walls

1. A consumable for a smoking substitute device, the consumable comprising:
a liquid reservoir defining a volume which contains vapourisable liquid, wherein the liquid reservoir contains less than 20% air by volume.

2. The consumable of claim 1, wherein the liquid reservoir contains less than 10% air by volume.

3. The consumable of claim 2, wherein the liquid reservoir contains less than 5% air by volume.

4. The consumable of claim 3, wherein the liquid reservoir contains less than 3% air by volume.

5. The consumable of claim 4, wherein the liquid reservoir contains less than 2% air by volume.

6. The consumable of claim 5, wherein the liquid reservoir contains less than 1% air by volume.

7. The consumable of claim 6, wherein the liquid reservoir contains substantially no air.

8. The consumable of claim 1, wherein the liquid reservoir contains 1.5 ml of vaporisable liquid.

9. The consumable of claim 1, further comprising a solid air-displacing element, located within the liquid reservoir, and which is configured to fill at least 8% of a volume of the liquid reservoir.

10. The consumable of claim 9, wherein the solid air-displacing element is configured to fill no more than 20% of the volume of the liquid reservoir.

11. The consumable of claim 9, wherein the solid air-displacing element is formed of silicone.

12. The consumable of claim 9, wherein the solid air-displacing element is toroidal in shape.

13. The consumable of claim 9, wherein the solid air-displacing element has a volume of greater than 0.13 ml and no more than 0.33 ml.

14. The consumable of claim 1, further comprising:
an outlet (306), at least partially located within the liquid reservoir; and
a heater assembly, at least partially located within the liquid reservoir and sealed to the outlet via a seal.

15. The consumable of claim 14, wherein the outlet fluidly connects the heater assembly to a mouthpiece located at an opposite end of the consumable to the heater assembly.

16. The consumable of claim 14, wherein the solid air-displacing element is toroidal in shape, and wherein the outlet is present within the hole of the toroidal shape solid air-displacing element.

17. The consumable of claim 14, further comprising a solid air-displacing element, located within the liquid reservoir, and which is configured to fill at least 8% of a volume of the liquid reservoir, and wherein the solid air-displacing element is located proximal to the heater assembly.

18. The consumable of claim 17, wherein the solid air-displacing element is the seal, sealing the outlet to the heater assembly.

19. The consumable claim 17, wherein the solid air-displacing element is located on an opposite side of the heater assembly to an air inlet of the consumable.

20. The consumable of claim 17, wherein the solid air-displacing element extends from the position proximal to the heater assembly along an axis of the outlet.

21. The consumable of claim 20, wherein the solid air-displacing element extends along at least 20% of the length of the outlet.

22. A smoking substitute device, including the consumable of claim 1.

23. A method of filling a consumable for a smoking substitute device, the method comprising:
adding vapourisable liquid to a liquid reservoir of the smoking substitute device, wherein after filing the consumable, the liquid reservoir contains less than 20% air by volume.

24. The method of claim 23, wherein the method of filling further comprises adding a solid air-displacing element to the liquid reservoir.

25. The method of claim 23, wherein the method comprises adding the vapourisable liquid before adding the solid air-displacing element.

26. The method of claim 23, wherein after filing the consumable, the liquid reservoir contains less than 10% air by volume.

27. The method of claim 26, wherein after filing the consumable, the liquid reservoir contains less than 5% air by volume.

28. The method of claim 27, wherein after filing the consumable, the liquid reservoir contains less than 3% air by volume.

29. The method of claim 28, wherein after filing the consumable, the liquid reservoir contains less than 2% air by volume.

30. The method of claim 29, wherein after filing the consumable, the liquid reservoir contains less than 1% air by volume.

31. A method of preparing a consumable for a smoking substitute device, the consumable comprising:
a liquid reservoir defining a volume which contains vapourisable liquid; and
a heater assembly comprising a wick; and
wherein the method comprises priming the consumable to force vapourisable liquid from the liquid reservoir into the wick.

32. The method according to claim 31, wherein the consumable comprises deformable walls, the priming the consumable comprising squeezing the deformable walls to force vapourisable liquid into the wick.

33. The method according to claim 32, wherein the liquid reservoir is a tank, the deformable walls forming walls of the tank.

34. The method according to claim 32, wherein the consumable comprises a button, wherein the priming comprises pushing the button to squeeze the deformable walls.

35. The method according to claim 34, wherein the button comprises a window permitting a level of vapourisable liquid in the liquid reservoir to be visually assessed.

36. The method according to claim 32, wherein the priming comprises squeezing the deformable walls at least three times.

37. The method according to claim 32, wherein the priming comprises squeezing the deformable walls when the consumable is connected to a main body of the smoking substitute device, the consumable and the main body configured to prevent oversqueezing of the deformable walls.

38. The method according to claim 31, wherein the liquid reservoir contains less than 5% air by volume.

39. The method according to claim 31, wherein the liquid reservoir contains less than 3% air by volume.

40. The method according to claim 31, wherein the liquid reservoir contains less than 1% air by volume.

41. The method according to claim 31, wherein the liquid reservoir contains substantially no air.

42. The method according to claim 31, wherein the liquid reservoir contains 1.5 ml of vaporisable liquid.

43. The method according to claim 31, wherein the consumable further comprises a solid air-displacing element, located within the liquid reservoir, and wherein the solid air-displacing element fills at least 8% of a volume of the liquid reservoir.

44. A consumable for a smoking substitute device, the consumable comprising:
a liquid reservoir defining a volume for storing vapourisable liquid;
a heater assembly comprising a wick; and
a priming device actuatable to force vapourisable liquid from the liquid reservoir into the wick.

45. A smoking substitute device comprising the consumable of claim 44.
